(19) United States
(12) Patent Application Publication
(10) Pub. No.: US 2013/0079371 A1
(43) Pub. Date: Mar. 28, 2013

(54) Bioadhesive Compositions of Local Anaesthetics

(71) Applicants: Mark Sundberg, Arsta (SE); Arne Brodin, Sodertalje (SE); Jonas Gustafsson, Uppsala (SE)

(72) Inventors: Mark Sundberg, Arsta (SE); Arne Brodin, Sodertalje (SE); Jonas Gustafsson, Uppsala (SE)

(73) Assignee: PHARMANEST AB, Solna (SE)

(21) Appl. No.: 13/638,519

(22) PCT Filed: Mar. 31, 2011
(86) PCT No.: PCT/EP11/55025

§ 371 Date: Dec. 12, 2012

Related U.S. Application Data
(60) Provisional application No. 61/325,425, filed on Apr. 19, 2010.

(30) Foreign Application Priority Data
Apr. 1, 2010 (SE) 1050322-5

(51) Int. Cl. A61K 47/14; A61K 31/167; A61L 2/00; A61K 47/44; A61K 47/26; A61K 31/445; A61K 31/245

(52) U.S. Cl. CPC A61K 47/14; A61K 31/445; A61K 31/167; A61K 31/245; A61K 47/44; A61K 47/26; A61L 2/0023
USPC 514/330; 514/626; 514/536; 422/26

(57) ABSTRACT

The present invention relates to a gelling bioadhesive pharmaceutical composition comprising one or more local anaesthetics in base form and which is suitable for topical administration. The compositions have anisotropic organic phase behaviour that admits swelling at administration site with excess water.

### FIELD OF THE INVENTION

The present invention relates to new long acting pharmaceutical compositions comprising local anaesthetics for topical administration. The pharmaceutical compositions can be used for reducing pain in connection with clinical conditions and clinical procedures.

### BACKGROUND TO THE INVENTION

Local anaesthetics are commonly used to inhibit nociceptive pain, and are usually administered by local injection. Pharmaceutical compositions for local injection normally contain local anaesthetics at a concentration of 1 to 2%.

In the preparation of pharmaceutical compositions for topical administration it is preferred to have the local anaesthetic present at a higher concentration.

Local anaesthetics of the amide type, ATC code N01BB, are weak bases with a pKₐ of around 8. Consequently, in an aqueous solution at neutral pH these local anaesthetics are mostly present in their acid form. However, the acid form is charged and therefore less suitable to pass through biological membranes. In pharmaceutical compositions for topical administration it is therefore preferred to have the local anaesthetic present in its base form which can readily pass through biological membranes. This can be achieved by adjusting the pH of the pharmaceutical compositions to a pH around or even preferably above the pKₐ of the local anaesthetic, i.e. to a pH above 8 or higher.

However, this leads to problems relating to the poor solubility and stability in aqueous solutions of the base form of the local anaesthetics.

This problem has been addressed for e.g. in EP 0833612 which discloses a pharmaceutical composition comprising an eutectic mixture of lidocaine base and prilocaine base. This mixture is in oil form at room temperature and can therefore be formulated as an emulsion. This eutectic mixture can only be obtained with a few local anaesthetics with different suitable melting points, exemplified by lidocaine base and prilocaine base. EP 1629852 describes a system where the local anaesthetic is kept in a solution at acidic pH and only mixed with a buffering solution with high pH shortly prior to use, providing a solution of the local anaesthetic at a pH between 5.5 and 7. In this pH interval only a small portion of the local anaesthetic is present in the base form, the form that readily penetrates membranes. There are numerous examples in the prior art of topical lipid based delivery systems that may be suitable to apply a local anaesthetic to the skin or the surface of the body., such as the systems disclosed in for example JP 2006335651; and US Patent Applications Nos. US 20080139392 and US 20090247494. However, none of these applications give any particular guidance to a composition of local anaesthetics that is particularly effective for a long acting anaesthetic effect also at a site inside the body where a number specific requirements need to be met in terms of administration, sterility, stability, safety and efficacy.

The present invention aims provide such pharmaceutical compositions comprising one or more local anaesthetics at sufficiently high concentration and at a sufficiently high pH useful also at internal body sites.

### DESCRIPTION OF THE INVENTION

Before the present invention is described, it is to be understood that the terminology employed herein is used for the purpose of describing particular embodiments only and is not intended to be limiting, since the scope of the present invention will be limited only by the appended claims and equivalents thereof.

It must be noted that, as used in this specification and the appended claims, the singular forms “a,” “an,” and “the” include plural referents unless the context clearly dictates otherwise.

Also, the term “about” is used to indicate a deviation of +/−2% of the given value, preferably +/−5%, and most preferably +/−10% of the numeric values, where applicable.

The present invention generally relates to stabilized aqueous pharmaceutical bioadhesive gelling compositions of an anaesthetically effective amount of one or more local anaesthetics which at least at their site of administration has an anisotropic organic phase behaviour that admits swelling of the compositions at administration sites with excess water, such as mucous membranes. The compositions comprise a monoglyceride or a diglyceride, or mixtures thereof, of a long chain fatty acid in an amount of between about 15 to about 70% by weight and a free long chain fatty acid in an amount of between about 5 to about 60% by weight.

The anisotropic organic phase behaviour of the inventive composition means that the compositions include an anisotropic, lyotropic, liquid crystalline phase. In order to be capable of swelling, the compositions include a hexagonal phase or a lamellar phase, or mixtures thereof. Within the context of the invention, the compositions can be designed to swell in excess water and establish an increase in bioadhesivity, suitably at a mucous membrane. Alternatively, the compositions can be administered in suitably swollen form to topical sites without the presence of excess water.

The compositions can further comprise solubilizers which is preferred, or even necessary to provide anaesthetically effective compositions for many local anaesthetics. In general terms these compositions are purposefully adapted to be stable systems of local anaesthetics, solubilizers, monoglyceride and/or diglyceride, fatty acid and water which retain stability without precipitation or degradation, also following high temperature sterilization (conventional autoclavation), while being suitably viscous to be administrable with conventional invasive devices such as a syringe with a cannula as fine as 15 Gauge at room temperature or with an administration tool having a tip with an inner diameter of about 1 to 2 mm. The compositions are capable of establishing adhesive gel characteristics at the administration site so a long acting anaesthetic effect can be maintained from the release of the anaesthetic agent(s) from the gelling composition. The inventive compositions are useful for conventional topical use on the body surface, but are especially adapted for providing a controlled long-acting anaesthetic effect at sites inside the body, exemplified by the cervix and the uterus.

The local anaesthetic to be used in the pharmaceutical compositions according to the invention can be any local anaesthetic. Preferably the local anaesthetic is a local anaesthetic of the amide type, ATC code N01BB or a local anaesthetic of the ester type, ATC code N01BA. Most preferably the local anaesthetic of the amide type is selected from lidocaine, prilocaine, mepivacaine, ropivacaine, bupivacaine, levobupivacaine. Most preferably the local anaesthetic of the ester type is selected from benzocaine, tetracaine and chloroprocaine.

The local anaesthetic to be used in the preparation of the pharmaceutical compositions according to the invention can be in the form of a base or the corresponding acid. If the acid form of the local anaesthetics is used, pH of the pharmaceutical compositions is adjusted by addition of a suitable amount of a base, e.g. NaOH (ag). In the preparation the local anaesthetic can also be in the form of a salt, such as hydrochloride, or in the form of a solvate, such as hydrate.

According to one embodiment the pharmaceutical composition according to the invention comprises one or more long acting local anaesthetic such as ropivacaine, bupivacaine, levobupivacaine.

According to another embodiment the pharmaceutical composition according to the invention comprises one or more short acting local anaesthetic such as lidocaine, prilocaine, mepivacaine.

An important feature of the present invention is the final pH-value of the pharmaceutical composition which is adjusted to a value where sufficient amounts of the local anaesthetic(s) are present in the uncharged base form. This feature is important to promote the penetration of the local anaesthetic into the tissue and consequently be able to exert the anaesthetic effect. That the pH is high enough so that a sufficient amount of the local anaesthetic is in its base form (close to or higher than the pKₐ of the local anaesthetics) is an advantage over a physiological pH (7.4) due to the promoted penetration of the uncharged base form.

Accordingly, the pH-value of the pharmaceutical composition is adjusted with suitable acid or base in such a way that the final pH-value for the composition is higher or equal to the pKₐ of the local anaesthetic minus 1.0, preferably the final pH-value for the composition is higher or equal to the pKₐ of the local anaesthetic minus 0.5, even more preferably the final pH-value for the composition is higher or equal to the pKₐ of the local anaesthetic.

If the composition comprises two or more local anaesthetics the final pH-value for the composition is adjusted in relation to the pKₐ of the local anaesthetic with the lowest pKₐ value.

TABLE 1
Examples of pKₐ for local anaesthetics

| Local anaesthetic | pKₐ |
|---|---|
| lidocaine | 7.9 |
| prilocaine | 7.9 |
| mepivacaine | 7.6 |
| ropivacaine | 8.1 |
| bupivacaine | 8.1 |
| levobupivacaine | 8.1 |

The mono- or diglycerides (or the mixture thereof) of the inventive compositions are glycerides of long chain fatty acids (generally C16 to C22). The fatty acids preferably prefereably comprise a single unsaturation and most preferably they are selected among oleic acid and ricinoleic acid. Most preferably to comprise the compositions are glycerol monooleate (monoolein) and glycerol dioleate. Many commercial brands of such lipids are not entirely pure and commercial monooleates may comprise low levels of diolein and triolein. Such brands are generally regarded as applicable with the present invention.

The fatty acid is preferably selected from long chain unsaturated fatty acids, preferably oleic acid, and ricinoleic acid. Most preferably the fatty acid is oleic acid.

Alternatively, the fatty acid can be selected among long-chain saturated fatty acids, most preferably the fatty acids are selected among palmitic acid and stearic acid.

Suitable solubilizers to comprise in the inventive compositions are of the polysorbate type, such as Tween 20, Tween 80; sorbitan fatty acid ester typ, such as Span 20, Span 80; Cremophors, such as Cremophor EL and glycerol formal. Preferably, the solubilizer is of the polysorbate type or a polyoxyethylated castor oil.

The total amount of monoglycerides or diglyceride and free fatty acids together in the composition is more suitably than 50% by weight in the composition, preferably between 50 to 75% by weight. The water content of the compositions is typically less than 50% by weight, suitably less than 30% by weight andpreferably, between 5 to 20% by weight.

The monoglycerides and/or diglyceride are preferably present in an amount of 20 to 50% by weight. The fatty acids are preferably present in an amount of between 15 to 70% by weight, preferably in an amount of between 25 to 50% by weight.

A certain embodiment provides a gel semi-solid or solid at 40° C. comprising lamellar and/or hexagonal phases, wherein the composition comprises ropivacaine in an amount of between 3 to 10% by weight; glycerol monooleate in an amount of between 40 to 70% by weight; oleic acid or ricinoleic acid in an amount of between 15 to 30% by weight; and a polysorbate type or polyoxyethylated castor oil type (Cremophor) solubilizer in an amount of between 10 to 20% by weight. The water is present in an amount between 10 to 20% by weight. Tween 80 is a suitable solubilizer. Suitably, these compositions have ratio of monooleate to oleic acid that is 40 to 60 (40/60) varying within the given concentration ranges.

In one suitable example proving solid gels at 40° C. including lamellar and/or hexagonal phases, the compositions include about 3% ropivacaine; about 42 to about 56% glycerol mono oleic acid; about 14 to about 29% by weight of oleic acid and about 10% by weigh polysorbate solubilizer (examplied by Tween 80) and between about 14 to about 18% by weight of water.

In another examples of this embodiment, where a stable gel including a lamellar phase have been established, the compositions comprise 10% by weight of ropivacaine with 5 to 10% by weight of polysorbate solubilizer (such as Tween 20) or sorbitan fatty acid esters (such as Span 20 or Span 80) or Cremphore type solubilizer (such as Cremophore EL) and 14 to 20% by weight of water.

Further preferred embodiments of the invention are pharmaceutical compositions comprising;
(a) a local anaesthetic selected from prilocaine, lidocaine, and tetracaine in an amount of between 1 to 20% by weight;
(b) one or more lipids selected from medium chain monoglycerides and glycerol monooleate in an amount of between 10 to 30% by weight;
(c) one or more fatty acids selected from oleic acid and ricinoleic acid in an amount of between 15 to 50% by weight; and
(d) glycerol formal in an amount of between 0 to 30% by weight.

Other preferred embodiments of the invention are pharmaceutical compositions comprising;
(a) a local anaesthetic selected from prilocaine, lidocaine, and tetracaine in an amount of between 1 to 20% by weight;
(b) one or more lipids selected from medium chain monoglycerides and glycerol monooleate in an amount of between 10 to 30% by weight;
(c) one or more fatty acids selected from oleic acid and ricinoleic acid in an amount of between 15 to 50% by weight; and
(d) Tween 80 in an amount of between 0 to 30% by weight; preferably in an amount of between 0 to 10% by weight.

According to another aspect, the invention relates to a method of preparing a gelling bioadhesive pharmaceutical composition capable of exerting a long term anaesthetic effect in an aqueous environment. The method comprises the consecutive steps of providing a mixture of a monoglyceride of long-chain unsaturated fatty acid, a free long-chain fatty acid and a solubilizer for a local anesthetic; adding a local anaesthetic to the mixture of the previous step; adding a water at a basic pH (suitably a pH about 8.0 to 8.5) to the mixture of the previous step; and thereby obtaining a gelling composition with an isotropic organic phase behaviour that admits swelling at an administration site with excess water. The local anaesthetic added to the start mixture can be in solid form or suitably dissolved in one of components of the start mixture. Preferably, monoglycerides and the fatty acid together are included to more than 50% by weight, preferably between 50 to 75% by weight, in the resulting composition; and wherein the water content is between 5 to 20% by weight in the resulting composition. The monoglyceride is preferably glycerol monooleate and the fatty acid is preferably oleic acid. The solubilizer preferably is of the polysorbate type or a polyoxyethylated castor oil and the local anaesthetic preferably is ropivacaine. The so described method can generally be followed to produce any of the earlier embodied compositions.

The pharmaceutical compositions according to the invention can be formulated for topical administration on any mucosal tissue, such as but not limited to, oral, nasal, intravaginal, intracervical, pericervical, intrauteral, intrarectal administration.

The pharmaceutical compositions according to the invention can be formulated for dermal administration on healthy, diseased and/or injured skin. Dermal administration can be made directly from the container, by hand, or by means of or together with patches, bandages and wound dressings.

The pharmaceutical compositions can be administrated by means of a syringe. The syringe can be further provided with an applicator. The applicator can be in the form of a tube.

The pharmaceutical compositions according to the present invention can be used for reducing pain in connection with various clinical conditions and clinical procedures.

Accordingly, in one aspect the present invention provides methods for reducing pain in connection with clinical conditions and clinical procedures comprising the administration of a pharmaceutical composition according to the invention.

Such clinical conditions are exemplified by, but not limited to, wound healing, especially burn wounds, skin ulcers, hemorrhoids, anal fissures; herpes zoster, herpes simplex infections, especially herpes labilalis, and herpes genitalis

Such clinical procedures are exemplified by, but not limited to, obstetric procedures, such as during labor, gynaecological procedures, such as application of intra uterine devices (IUD), hysteroscopy, in vitro fertilization, spontaneous and legal abortions, and general vaginal examination, dental procedures, surgical procedures, such as skin grafting.

The methods can comprise administration on any mucosal tissue, such as but not limited to, oral, nasal, intravaginal, intracervical, pericervical, intrauteral, intrarectal administration.

The methods can comprise dermal administration on healthy, diseased and/or injured skin. Dermal administration can be made directly from the container, by hand, or by means of or together with patches, bandages and wound dressings.

The administration can be made by means of a syringe. The syringe can be further provided with an applicator. The applicator can be in the form of a tube.

The bioadhesive pharmaceutical compositions according to the invention are generally capable of attaching to a mucous surface in the process described as mucoadhesion. This process involves spreading, wetting and swelling of the pharmaceutical compositions at the mucous surface, initiates intimate contact between the components of the pharmaceutical compositions and the mucus layer. Interdiffusion and interpenetration take place between the components of the pharmaceutical compositions and the mucus gel network, creating a greater area of contact. Entanglements and secondary chemical bonds are formed between the components of the pharmaceutical compositions and the mucin molecules. The components of the mucus involved in interactions are the mucin molecules. These are glycoproteins of high molecular weight, which are also responsible for the viscoelastic properties of the mucus. The mucins are negatively charged at physiological pH because of sialic acid residues in the oligosaccharide units. Hydrogen bonds are often considered to be the most important of the types of secondary chemical bonds that can be formed in the mucoadhesion process. Other types of bonds that might be involved include ionic bonds and van der Waals interactions.

According to still another aspect, the present invention relates to a method of manufacturing a stabilized local anaesthetic product with such a low level of viable microorganisms that the product is suitable for topical administration to an internal body site. The method comprises a first step of providing a composition of a local anaesthetic in a concentration of between 1 to 10% by weight and solubilized with at least 5% of a solubilizer, the composition further comprising at least 50% by weight of a monoglyceride or a diglyceride, or mixtures thereof of together with a long chain free fatty acid. Preferably, the monoglycerides and the fatty acid together is included to more than 50% by weight, preferably between 50 to75% by weight, in the resulting composition; and wherein the water content is less than 50% by weight, preferably between 5 to 20% by weight in the resulting composition. Most preferably, the monoglycerides are glycerol monooleate and the fatty acid is oleic acid.

The following steps of the method relates to preparing a sealed container comprising the composition; subjecting the container with the composition to heat sterilization (autoclavation) less than 120° C., preferably below 115° C. and most preferably at about 105° C. for about 10 minutes; and finally obtaining a local anaesthetic product with maintained gelling characteristics and with so low level of viable microorganisms that the product is suitable for topical administration to an internal body site.

Any of the earlier disclosed or embodied gelling compositions with anisotropic lyotropic, liquid crystalline behaviour can be employed with this production method. It is of considerable advantage that the compositions of the present invention can be sterilized to an acceptable product at less harsh conditions than at autoclavation at 121° C. during 15 minutes, as otherwise expected/required by clinical authorities as it significantly reduces the risk for potentially harmful degradation products. It is contemplated that the systems components may synergistically contribute to an antimicrobial effect under the conditions of the method.

The compositions of the invention as described generally and in certain embodiments in the foregoing sections exhibit excellent stability even if subjected to harsh sterilization conditions. They generally include lamellar and/or hexagonal phases or in certain embodiments have the behaviour of a lamellar gel that is gelling in an aqueous environment such as at mucous membrane. The compositions are suitably cohesive or semisolid or solid with bioadhesive characteristics so they correctly remain at the administration site to exert the desired predetermined anaesthetic effect. These and other advantages will be demonstrated in the following experimental section.

### DESCRIPTION OF THE FIGURE

FIG. 1 is a graph illustrating the in-vitro release of ropivacaine from pharmaceutical compositions. Composition according to Table 14. -□- sample 1; -▪- sample 2; -Δ- sample 3; -▴- sample 4; -⋄- sample 5; -♦- sample 6; -◯- sample 7; -- sample 8; -*- sample 9.

FIG. 2 is shows mucoadhesive measurements for 3% ropivaciane lamellar gel formulations with different water concentrations.

### EXAMPLES

Aggregation structures that are formed in the presence of fatty acid and glycerol monooleate/glycerol dioleate/glycerol trioloeate were investigated as a means for preparing pharmaceutical compositions comprising local anaesthetics. A range of phase structures are possible with these systems.

Materials

Anaesthetics

Ropivacaine (base form)—Ropivacaine HCl was supplied by Chemos GmbH, Regenstauf, Germany. The HCl was dissolved in water and pH adjusted to pH>8 by addition of 1 M NaOH, and subsequently the precipitated base was collected by filtration

Tetracaine (base form)—Sigma-Aldrich (≧98%)

Benzocaine (base form)—Sigma (99%)

Lidocaine (base form)—Apoteket Produktion & Laboratorier (Eur. Kval. E.)

Lipids

GMO (glycerol monooleate)—Danisco, RYLO MG19 Pharma (melting point ˜40° C.)

Technical GMO—Aldrich (total impurities: 20-40% diglycerides, 20-40% triglycerides)

MCM (medium-chain monoglyceride)—AarhusKarlshamn Sweden AB, Karlshamn, Sweden

GDO (glycerol monooleate)—Danisco, Rylo DG19 Pharma

GMS (glycerol monostearate)—Danisco, Rylo MG19 Pharma

GML (glycerol monolinoleate—Danisco, Rylo MG13 WA Pharma

Organic acids

Oleic acid—Aldrich (puriss)

Ricinoleic acid—Aldrich (tech. 80%)

Palmitic acid—Sigma (Sigma grade)

Steraic acid—Sigma (99%)

Other Excipients Used in the Formulations

Glycerol formal—Fluka 98.0%)

Non-ionic surfactant, Tween 80 (Polysorbate 80)—Sigma-Aldrich

Non-ionic surfactant, Tween 20 (Polysorbate 20)—Sigma-Aldrich

Non-ionic surfactant. Span 80 (sorbitan fatty acid ester 80)—Sigma-Aldrich

Non-ionic surfactant. Span 20 (sorbitan fatty acid ester 20)—Sigma-Aldrich Sodium hydroxide (aq)—1-5 M

Method for Preparing Pharmaceutical Compositions.

Order of mixing of the different excipients (general procedure for all the gel formulations):
i. Melting of lipid (only glycerol mono and/or dioleate, glycerol monostearate, glycerol monolinoleate)
ii. Mixing of lipid and organic acid
iii. Addition, if necessary, of other excipients: glycerol formal or Tween 80
iv. Addition of ropivacaine
v. Stirring of solution until full dissolution
vi. Addition of certain amount of water (approximately 10%) was added to the solution by adding a sodium hydroxide solution with gentle stirring. pH of the solution containing ropivacaine was adjusted to pH 8.5.
vii. In some cases more water (pure Milli-Q water) was added to study the gelling behavior of the extra water addition.

### Example 1

### Formulations Using Lyotropic Phases

The initial tests in Table 2 with the lyotropic phase systems were made in order to establish the feasibility of this approach. It was found that by mixing glycerol monooleate (GMO), oleic acid and water a gel (very likely a cubic phase) was formed. Formulations were prepared where ropivacaine was mixed with GMO, oleic acid and water and a white gel was formed.

TABLE 2
Initial tests for the lyotropic phase systems.
Water addition refers to the addition of NaOH (aq) for adjustment
to pH 8.5 for the compositions containing local anesthetics.

| | | Oleic | Glycerol | | | |
|---|---|---|---|---|---|---|
| Ropivacaine | GMO | acid | formal | Water | | |
| (%) | (%) | (%) | (%) | (%) | pH | Appearance |
| — | 45 | 45 | — | 10 | 5 | Gel |
| — | 35 | 35 | — | 30 | 2.6 | Gel |
| — | 25 | 25 | — | 50 | 1 | Gel |
| 8 | 41 | 41 | 10 | — | | Clear, viscous |
| | | | | | | solution |
| 5 | 32.5 | 32.5 | 30 | — | | Clear, viscous |
| | | | | | | solution |
| 8 | 21 | 21 | 50 | — | | Clear solution |
| 5 | 42.5 | 42.5 | — | 10 | | Clear, viscous |
| | | | | | | solution (pH 9) |
| 5 | 37.5 | 37.5 | — | 20 | | White gel (pH 9) |
| 5 | 32.5 | 32.5 | — | 30 | | White gel (pH 9) |

### Example 2

### Formulations with GMO and Oleic Acid

The composition ranges of the different excipients are coupled to the amount of ropivacaine in the formulation. In Table 3, formulations with different ropivacaine concentrations are presented. The table is sorted after increasing ropivacaine concentration in the formulation. Different combinations of the components offered a gel formulation where ropivacaine was solubilized. The phase behavior of the formulations was investigated with cross-polarizers to distinguish between lamellar and cubic phases in the gel formulation.

TABLE 3A
Ropivacaine, lipid - GMO, organic acid - oleic acid
Formulations investigated for in-situ gelling

| | | Oleic | Glycerol | | |
|---|---|---|---|---|---|
| Ropivacaine | GMO | acid | formal | Water | |
| (%) | (%) | (%) | (%) | (%) | Appearance |
| 4 | 25 | 25 | 23 | 23 | Viscous, white |
| 6 | 24 | 24 | 23 | 23 | Gel (cubic) |
| 7 | 26 | 26 | 25 | 17 | Gel (cubic) |
| 7 | 37 | 37 | 9 | 9 | Viscous, clear |
| | | | | | solution |
| 7 | 28 | 28 | 27 | 9 | Clear solution |
| 7 | 19 | 19 | 45 | 9 | Clear solution |
| 10 | 21 | 49 | 10 | 10 | Clear solution |
| 10 | 18 | 42 | 10 | 20 | Clear, viscous |
| | | | | | solution |
| 10 | 29 | 44 | 0 | 17 | Clear, lamellar gel |
| 11 | 9 | 65 | 6 | 9 | Clear, viscous |
| 15 | 26 | 39 | 10 | 10 | Clear solution, pH 8 |
| 15 | 26 | 39 | 10 | 10 | Viscous, clear |
| | | | | | solution, pH 8.5 |
| 15 | 22 | 33 | 10 | 20 | Clear gel, pH 8.0 |
| 15 | 22 | 33 | 10 | 20 | Clear gel, pH 8.1 |
| 15 | 22 | 33 | 10 | 20 | Clear gel, pH 8.5 |
| 15 | 17 | 25 | 10 | 33 | Clear gel |
| 15 | 10 | 15 | 10 | 50 | Clear gel |
| 15 | 18 | 27 | 30 | 10 | White solution |
| 15 | 18 | 27 | 30 | 10 | White solution |
| 15 | 33 | 33 | 10 | 10 | Viscous, white |
| 15 | 23 | 23 | 30 | 10 | White solution |
| 15 | 8 | 62 | 5 | 9 | Clear, viscous |
| 16 | 19 | 44 | 4 | 17 | Lamellar gel |
| 16 | 20 | 46 | 9 | 9 | Clear, slightly viscous |
| 16 | 13 | 52 | 9 | 9 | Clear, slightly viscous |
| 17 | 21 | 48 | 5 | 9 | Clear, slightly viscous |
| 18 | 22 | 50 | 10 | 0 | Clear solution |
| 19 | 23 | 53 | 5 | 0 | Clear solution |

It should be noted that pH has a dramatic effect on the viscosity of the formulations, where a higher pH closer to pH 9 increases the viscosity. Both pH and the amount of water added to the formulation can be used as a tool to obtain a ropivacaine formulation with the desired gelling behaviour. The content of water in the formulation can be rather low to obtain a low-viscosity formulation to be easily applied during the application on the mucosal surface. Nevertheless, the viscosity of the formulation should be high enough to ensure that the formulation adheres to the mucosal surface. When the formulation adheres to the mucosal surface it can absorb more water and form a more rigid gel, which will further promote the adhesion to the mucosal surface. The increased gel strength by high water concentration has been confirmed by preparing formulations with the same ropivacaine concentration but varying the amount of water. Gel samples with high concentration of water (up to 50%) are much more rigid than the samples with lower concentration of water (10%) which are present as a viscous solution.

TABLE 3B
Ropivaciane with varying concentrations of oleic acid

| Sample no | | | | | | |
|---|---|---|---|---|---|---|
| 3% | | Oleic | Tween | | Konc | |
| ropivacain, | GMO | Acid | 80 | Water | NaOH | |
| Oleic acid | (%) | (%) | (%) | (%) | (M) | Results |
| 11-43b | 64.6 | 7.3 | 10.3 | 14.7 | 0.82 | Lamellar |
| | | | | | | solution |
| | | | | | | Less viscous |
| | | | | | | at 40° C. |
| 11-43 | 66.2 | 7.5 | 10.5 | 12.6 | 0.88 | Lamellar |
| | | | | | | solution |
| | | | | | | Less viscous |
| | | | | | | at 40° C. |
| 11-40 | 55.9 | 14.0 | 10.0 | 17.1 | 1.50 | Lamellar gel |
| | | | | | | Solid at 40° C. |
| 11-41b | 42.4 | 28.3 | 10.1 | 16.1 | 1.30 | Lamellar gel |
| | | | | | | Solid at 40° C. |
| 11-41 | 43.1 | 28.8 | 10.3 | 14.7 | 1.36 | Lamellar gel |
| | | | | | | Solid at 40° C. |
| 11-42b | 28.0 | 42.0 | 10.0 | 17.0 | 1.17 | Lamellar |
| | | | | | | solution |
| | | | | | | Less viscous |
| | | | | | | at 40° C. |
| 11-42 | 28.0 | 42.1 | 10.0 | 16.8 | 1.50 | Lamellar |
| | | | | | | solution |
| | | | | | | Less viscous |
| | | | | | | at 40° C. |

### Example 3

### Formulations with Replacement of GMO with Other Lipids

Formulations were prepared where GMO were replaced with technical GMO and other lipids as specified below. The composition ranges of the different excipients are coupled to the amount of ropivacaine in the formulations. The content of the formulations that were prepared are listed in Tables 4-6. All the investigated lipids offered the possibility to form gel formulations of both lamellar and cubic phase structure. This enables flexibility in the choice of components to be used in the formulation since all the lipids used within this study offered the possibility to form a gel.

TABLE 4
Ropivacaine, lipid - technical GMO, organic acid - oleic acid
Formulations investigated for in-situ gelling

| | | Oleic | Glycerol | | | |
|---|---|---|---|---|---|---|
| Ropivacaine | Technical | acid | formal | Water | NaOH | |
| (%) | GMO (%) | (%) | (%) | (%) | (M) | Appearance |
| 9 | 29 | 44 | 9 | 9 | 1 | Clear solution |
| 9 | 25 | 38 | 9 | 18 | 2.4 | Turbid (lamellar) gel |
| 9 | 25 | 38 | 9 | 18 | 0.7 | Turbid (not lamellar) gel |
| 9 | 22 | 33 | 9 | 27 | 0.7 | Turbid (lamellar) gel |
| 10 | 15 | 23 | 5 | 48 | 1.2 | Turbid (partly lamellar) |
| | | | | | | gel |

In Tables 5 and 6 GDO was used together with a different brand GMO:

GMO—glycerol monoleate (Rylo MG 19, min. 96% monoglycerides, max. 4& diglycerides)

GDO—glycerol dioleate (Rylo DG 19 Pharma, min. 94%, diglycerides, max. 1% monoglycerides, triglycerides max. 5%),

TABLE 5

| | | Oleic | | | | |
|---|---|---|---|---|---|---|
| Ropivacaine | | acid | Tween 80 | Water | NaOH | |
| (%) | | (%) | (%) | (%) | (M) | Appearance |
| | GMO/GDO | | | | | |
| | 40/60 (%) | | | | | |
| 2.8 | 26.9 | 40.4 | 9.4 | 20.4 | 1.98-2.20 | Solution with mixture |
| | | | | | | lamellar and cubic |
| | | | | | | phases |
| 9.5 | 25.3 | 37.8 | 9.8 | 17.6 | 1.47-2.13 | Solution with mixture |
| | | | | | | lamellar and cubic |
| | | | | | | phases |
| | GMO/GDO | | | | | |
| | 60/40 (%) | | | | | |
| 2.9 | 27.4 | 41.1 | 9.5 | 19.1 | 0.7 | Solution with mixture |
| | | | | | | lamellar and cubic |
| | | | | | | phases |
| 9.3 | 24.8 | 36.9 | 9.5 | 19.4 | 1.2 | Solution with mixture |
| | | | | | | lamellar and cubic |
| | | | | | | phases |

TABLE 6

| | | Oleic | Tween | | | |
|---|---|---|---|---|---|---|
| Ropivacaine | GDO | acid | 80 | Water | NaOH | |
| (%) | (%) | (%) | (%) | (%) | (M) | Appearance |
| 2.9 | 28.2 | 42.3 | 9.8 | 16.8 | 1.9-2.37 | Solution |
| | | | | | | with mixture |
| | | | | | | lamellar |
| | | | | | | and cubic |
| | | | | | | phases |
| 9.7 | 25.6 | 25.6 | 9.8 | 16.7 | 1.65-2.68 | Solution |
| | | | | | | with mixture |
| | | | | | | lamellar |
| | | | | | | and cubic |
| | | | | | | phases |

### Example 4

### Replacement of Oleic Acid From the Original Ropivacaine Formulation

Formulations were prepared where oleic acid was replaced with ricinoleic acid. A lipid (GMO or lecithin) was mixed with ricinoleic acid followed by addition of glycerol formal and ropivacaine and the formulation were evaluated, see Tables 7 and 8. The composition ranges of the different excipients are coupled to the amount of ropivacaine in the formulation. Ricinoleic acid was successfully used in the formulations. Combining the results presented in this example with the results presented in Example 3 (studying different lipids), it is shown that a flexible formulation recipe is developed where different combinations of lipids and organic acids with ropivacaine can be used and still obtain a formulation with gelling behaviour.

TABLE 7
Ropivacaine, lipid - GMO, organic acid - ricinoleic acid
Formulations investigated for in-situ gelling

| Ropivacaine | GMO | Ricinoleic | Glycerol | Water | |
|---|---|---|---|---|---|
| (%) | (%) | acid (%) | formal (%) | (%) | Appearance |
| 9 | 29 | 44 | 9 | 9 | Clear, low- |
| | | | | | viscous |
| 9 | 25 | 38 | 9 | 18 | Clear, slightly |
| | | | | | viscous |
| 9 | 20 | 30 | 9 | 32 | Lamellar gel |
| 10 | 32 | 48 | 0 | 10 | Clear, low- |
| | | | | | viscous |
| 10 | 28 | 42 | 0 | 20 | Clear, low- |
| | | | | | viscous |
| 10 | 24 | 36 | 0 | 30 | Lamellar gel |

TABLE 8
Ropivacaine formulations with varying concentrations of ropivaciane with
different fatty acids and water concentration.

| | | Free | | | | |
|---|---|---|---|---|---|---|
| | GMO | Fatty | Tween | Water | Conc | |
| Sample no | (%) | Acid (%) | 80 (%) | (%) | NaOH (M) | Results |
| 3% | | | | | | |
| ropivacain, | | | | | | |
| Ricinoleic | | | | | | |
| acid | | | | | | |
| 11-44 | 37.2 | 24.8 | 8.9 | 26.5 | 1.47 | Lamellar solution |
| | | | | | | Less viscous at 40° C. |
| 3% | | | | | | |
| ropivacain, | | | | | | |
| Palmitic | | | | | | |
| acid | | | | | | |
| 11-45b | 59.7 | 15.0 | 10.7 | 11.4 | 0.90 | Lamellar, turbid |
| | | | | | | solution |
| | | | | | | Less viscous at 40° C. |
| 11-45 | 61.2 | 15.3 | 77.0 | 9.2 | 1.00 | Lamellar, turbid |
| | | | | | | solution |
| 11-46 | 37.7 | 25.1 | 9.0 | 25.5 | 0.79 | Lamellar |
| | | | | | | solution/gel |
| | | | | | | Solid at 40° C. |
| 11-47 | 23.5 | 35.2 | 8.5 | 30.4 | 1.05 | Lamellar, turbid |
| | | | | | | solution/gel |
| 11-47c | 25.1 | 37.6 | 9.1 | 25.3 | 0.78 | Lamellar (?) |
| | | | | | | solution/gel, turbid |
| 11-47b | 29.3 | 44.0 | 10.6 | 12.9 | 1.73 | Solid, not lamellar |
| 8% | | | | | | |
| ropivacain, | | | | | | |
| Palmitic | | | | | | |
| acid | | | | | | |
| 11-48b | 57.5 | 14.5 | 11.6 | 8.4 | 1.28 | Lamellar, turbid |
| | | | | | | solution |
| | | | | | | Less viscous at 40° C. |
| 11-48c | 58.6 | 14.8 | 11.8 | 6.7 | 1.50 | Lamellar, turbid |
| | | | | | | solution |
| 11-49 | 32.5 | 21.6 | 8.6 | 28.7 | 0.85 | Soft cream |
| | | | | | | (lamellar?) |
| 12% | | | | | | |
| ropivacain, | | | | | | |
| Palmitic | | | | | | |
| acid | | | | | | |
| 11-48 | 60.9 | 15.3 | 12.1 | — | — | ropivacain did not |
| | | | | | | dissolve |
| 3% | | | | | | |
| ropivacain, | | | | | | |
| Stearic acid | | | | | | |
| 11-51 | 38.9 | 25.9 | 9.2 | 23.1 | 1.14 | Soft white cream |
| | | | | | | (lamellar?) |
| 11-55 | 23.2 | 34.5 | 8.5 | 31.4 | 1.63 | Solid white cream |
| | | | | | | (lamellar?) |
| 11-56 | 9.1 | 35.5 | 6.4 | 47.2 | 1.91 | Solid white cream |
| | | | | | | (lamellar?) |
| 11% | | | | | | |
| ropivacain, | | | | | | |
| Stearic acid | | | | | | |
| 11-54 | 40.5 | 26.9 | 10.7 | 11.2 | 1.31 | Solid cream |
| | | | | | | (lamellar?) |

Suitable compositions from Table 8A include:

Oleic Acid

3% ropivacain: lamellar gel with 14-29% oleic acid (10% Tween 80, water concentration: 15-17%)

3% ropivacain: mixture of lamellar and cubic phases with 7% oleic acid (10% Tween 80,

water concentration: 13-14%)

3% ropivacain: mixture of lamellar and cubic phases with 40% oleic acid (10% Tween 80,

water concentration: 17%)

10% ropivacain: lamellar gel with 38-42% oleic acid (10% Tween 80, water concentration: 10-25%)

Ricinoleic Acid

3% ropivacain: mixture of lamellar and cubic phases with 25% ricinoleic acid (10% Tween 80, water concentration: 27%)

Palmitic Acid

3% ropivacain: lamellar gel with 25-35% palmitic acid (10% Tween 80, water concentration: 25-30%)

8% ropivacain: mixture of lamellar and cubic phases with 15% palmitic acid (10% Tween 80,

water concentration: 7-8%)

12% ropivacain: precipitation 15% palmitic acid (10% Tween 80)

Stearic Acid

3% ropivacain: white cream, mixture of lamellar and cubic phases with 25-35% stearic acid (10% Tween 80, water concentration: 23-47%)

11% ropivacain: solid white cream, mixture of lamellar and cubic phases with 27% stearic acid, 10% Tween 80, water concentration: 11%)

### Example 5

### Ropivacaine Formulations with Varying Concentrations and Different Solubilizers

A surfactant, Tween 80 was added to formulations with ropivacaine, GMO and oleic acid to improve the phase stability of the gel formulation. To confirm the improved stability of the formulations containing Tween 80, two gels with/without Tween 80 were added to a buffer solution (pH 7.4, 0.9% NaCl). The gel containing Tween 80 did not dissolve in the buffer, while the sample without Tween 80 dissolved in the buffer. This suggested that Tween 80 has the capability to stabilize the gel formulation. Formulations with Tween 80 are presented in Table 9A and it was shown to be possible to form a lamellar type of gel formulation. Table 9B demonstrates the efficacy of other solubilizers. It was found that it was possible to exclude glycerol formal when Tween 80 was present in the formulation recipe.

TABLE 9A
Ropivacain formulations with varying ropivacain-concentration,
concentration of Tween 80 and concentration of water
(the ratio of GMO/oleic acid was 40/60 is all samples).

| | | Oleic | | | Conc | |
|---|---|---|---|---|---|---|
| Sample | GMO | Acid | Tween | Water | NaOH | |
| no | (%) | (%) | 80 (%) | (%) | (M) | Results |
| 3% | | | | | | |
| ropivacain | | | | | | |
| 11-04 | 28.0 | 42.0 | 10.0 | 17.0 | 1.29 | Lamellar gel |
| 11-04b | 28.0 | 42.0 | 10.0 | 17.0 | 1.45 | Lamellar and |
| | | | | | | viscous |
| 11-05 | 26.0 | 38.9 | 15.2 | 17.0 | 1.55 | Lamellar and |
| | | | | | | viscous |
| 11-05b | 25.9 | 38.9 | 15.5 | 16.9 | 1.32 | Lamellar and |
| | | | | | | viscous |
| 11-06 | 23.1 | 34.7 | 19.9 | 19.3 | 1.65 | Lamellar and |
| | | | | | | viscous |
| 11-07* | 19.4 | 29.0 | 29.0 | 19.7 | 1.58 | Not lamellar, |
| | | | | | | solution |
| 11-07b | 19.8 | 29.7 | 29.7 | 17.9 | 1.60 | Not lamellar, |
| | | | | | | solution |
| 11-07 | 20.0 | 30.0 | 30.0 | 17.0 | 0.88 | Not lamellar, |
| | | | | | | solution |
| 11-17 | 16.1 | 24.2 | 40.5 | 16.1 | 1.90 | Not lamellar, |
| | | | | | | solution |
| 9% | | | | | | |
| ropivacain | | | | | | |
| 11-02 | 18.7 | 28.1 | 17.7 | 26.6 | 1.38 | Lamellar |
| 10% | | | | | | |
| ropivacain | | | | | | |
| 10-01 | 28.0 | 42.0 | 10.0 | 10.0 | 1.90 | Clear, low- |
| | | | | | | viscous solution |
| 10-02 | 26.0 | 39.0 | 10.0 | 15.0 | 1.60 | Clear, viscous |
| | | | | | | solution |
| 10-03 | 25.0 | 38.0 | 10.0 | 17.0 | 1.80 | Lamellar gel |
| 11-10 | 25.9 | 38.9 | 10.2 | 24.9 | 1.07 | Lamellar and |
| | | | | | | viscous |
| 11-01b | 22.8 | 34.2 | 14.5 | 19.0 | 1.61 | Not lamellar, |
| | | | | | | solution |
| 11-01 | 22.9 | 34.4 | 14.8 | 18.2 | 2.34 | Lamellar gel |
| 11-01b | 23.3 | 35.0 | 14.8 | 17.1 | 1.01 | Not lamellar, |
| | | | | | | solution |
| 11-02b | 20.9 | 31.3 | 19.7 | 17.5 | 1.95 | Not lamellar, |
| | | | | | | solution |
| 11-02b | 21.1 | 31.7 | 20.0 | 17.1 | 1.32 | Lamellar and |
| | | | | | | viscous (less |
| | | | | | | cloudiness |
| | | | | | | than 11-02) |
| 11-03 | 16.5 | 24.8 | 28.9 | 20.2 | 1.97 | Lamellar and |
| | | | | | | viscous (with |
| | | | | | | precipitation) |
| 11-03b | 17.1 | 25.7 | 30.1 | 16.9 | 0.93 | Lamellar gel |
| | | | | | | (precipitation?) |
| 11-03b | 17.4 | 26.1 | 30.6 | 15.7 | 1.02 | lamellar gel |
| | | | | | | with less |
| | | | | | | precipitation |
| | | | | | | than 11-03 |
| 11-33 | 12.8 | 19.2 | 58.4 | 0.0 | — | Not dissolved |
| 15% | | | | | | |
| ropivacain | | | | | | |
| 11-08b | 23.0 | 34.5 | 9.9 | 17.8 | 1.30 | Not lamellar, |
| | | | | | | solution |
| 11-08b | 23.2 | 34.8 | 10.0 | 17.0 | 0.79 | Precipitation? |
| 11-08 | 23.2 | 34.7 | 10.1 | 17.3 | 1.76 | Not lamellar, |
| | | | | | | solution |
| 11-08 | 23.2 | 34.9 | 10.2 | 17.0 | 1.19 | Precipitation? |
| 11-08 | 24.1 | 36.1 | 10.5 | 14.0 | 1.50 | Not lamellar, |
| | | | | | | solution |

TABLE 9B
Formulations with 10% (wt) of ropivaciane with different
solubilizers (the ratio of GMO/oleic acid was 40/60 is all samples)

| Sample no | | | | | | |
|---|---|---|---|---|---|---|
| 10% | | | | | | |
| ropivacain | | Oleic | | Wa- | Conc | |
| (other | GMO | Acid | Solubilizer | ter | NaOH | |
| solubilisers) | (%) | (%) | (%) | (%) | (M) | Results |
| 11-12 | 24.7 | 37.1 | 9.8 | 18.6 | 1.16 | Lamellar and |
| | | | Span 80 | | | viscous |
| 11-10 | 25.2 | 37.8 | 10.2 | 16.9 | 0.88 | Lamellar and |
| | | | Span 20 | | | viscous |
| 11-11 | 26.2 | 39.3 | 10.4 | 13.6 | 0.64 | Lamellar and |
| | | | Cremophor | | | viscous |
| | | | EL | | | |
| 11-14 | 27.3 | 40.9 | 5.0 | 17.0 | 1.90 | Lamellar and |
| | | | Tween | | | viscous |
| | | | 20 | | | |
| 11-13 | 25.2 | 37.7 | 10.1 | 17.0 | 0.90 | Not lamellar, |
| | | | Tween | | | solution |
| | | | 20 | | | |
| 11-13b | 24.7 | 37.1 | 9.8 | 18.4 | 17.1 | Not lamellar, |
| | | | Tween | | | solution |
| | | | 20 | | | |

Suitable compositions from Table 9B include:

Tween 80

3% ropivacain: lamellar gel with 10-20% Tween 80 (water concentration: 17%)

10% ropivacain: lamellar gel with 10-20% Tween 80 (water concentration: 17-25%)

10% ropivacain: precipitation with 30% Tween 80 (water concentration: 16-20%)

15% ropivacain: no lamellar solution with 10% Tween 80 (water concentration: 14%)

15% ropivacain: precipitation with 10% Tween 80 (water concentration: 17%)

Concentration ranges:

3-10% ropivacain (15% ropivacain precipitation)

10-20% Tween 80

>17% water concentration

Tween 20

10% ropivacain: lamellar gel with 5% Tween 20 (water concentration: 17%)

Span 20

10% ropivacain: lamellar gel with 10% Span 20 (water concentration: 17%)

Span 80

10% ropivacain: lamellar gel with 10% Span 80 (water concentration: 19%)

Cremophor EL (Polyoxyl 35 Castor Oil)

10% ropivacain: lamellar gel with 10% Cremophor EL (water concentration: 14%)

### Example 6

### Formulations Using Lyotropic Phases with Other Local Anaesthetics

Three additional local anaesthetics were investigated in this study with the similar formulation procedure as for ropivacaine , i.e., mixing a lipid and an organic acid, followed by addition of other excipients (glycerol formal, Tween 80), ropivacaine and water.

In Table 10, the formulations with lidocaine (5% and 10%) are shown. Lidocaine has similar pKa as ropivacaine and the formulation recipe was therefore transferable to a lidocaine gel formulation. It should be noted that glycerol formal was excluded but it was still possible to obtain a lamellar gel.

TABLE 10
Lidocaine, lipid - GMO, organic acid - oleic acid
Formulations investigated for in-situ gelling

| Lidocaine | GMO | Oleic acid | Water | NaOH | |
|---|---|---|---|---|---|
| (%) | (%) | (%) | (%) | (M) | Appearance |
| 10 | 34 | 51 | 5 | | Clear solution |
| 5 | 34 | 51 | 10 | 2.4 | Lamellar gel |

Tetracaine and benzocaine are two local anaesthetics containing ester groups, which may hydrolyze in the presence of water. For formulations with tetracaine and benzocaine it is therefore desirable to minimize the amount of water present in formulation. Tetracaine has similar pKa as ropivacaine and could easily be formulated with a similar formulation as for ropivacaine, see Table 11. A lamellar type of gel with tetracaine was formed.

TABLE 11
Tetracaine, lipid - GMO, organic acid - oleic acid
Formulations investigated for in-situ gelling

| | | Oleic | | | | |
|---|---|---|---|---|---|---|
| Tetracaine | GMO | acid | Glycerol | Water | NaOH | |
| (%) | (%) | (%) | formal (%) | (%) | (M) | Appearance |
| 10 | 28 | 42 | 10 | 10 | 2.3 | Clear |
| | | | | | | solution |
| 10 | 23 | 35 | 10 | 22 | 3 | Lamellar, |
| | | | | | | viscous |
| | | | | | | (pH 8.5) |
| 10 | 29 | 43 | 10 | 8 | 2 | Lamellar gel |

### Example 7

### Mucoadhesion

Some samples were selected for qualitative evaluation of the mucoadhesion on a soaked dish cloth. Two types of behaviours of the gels on the dish cloth could be distinguished, either the gel was present on the surface or it was soaked into the dish cloth. When the gel was present on the surface it was adhering quite well and did not slide off when leaning the dish cloth. The samples that were soaked into the dish cloth were generally less viscous than the samples that were staying on the surface of the dish cloth. In Table 12, the results of the mucoadhesion tests are summarized.

TABLE 12
Mucoadhesion tests on formulations.

| | | Oleic | Glycerol | | | |
|---|---|---|---|---|---|---|
| Ropivacaine | Lipid (%) | acid | formal | Water | | |
| (%) | GMO | (%) | (%) | (%) | Appearance | Mucoadhesion |
| 10 | 21 | 49 | 10 | 10 | Clear solution | Soaked into the |
| | | | | | | dish cloth |
| 10 | 18 | 42 | 10 | 20 | Clear, viscous | Soaked into the |
| | | | | | solution | dish cloth |
| 10 | 29 | 44 | 0 | 17 | Clear, | Soaked into the |
| | | | | | lamellar gel | dish cloth |
| 15 | 26 | 39 | 10 | 10 | Clear solution | Gel on the |
| | | | | | (pH 8) | surface of the |
| | | | | | | dish cloth |
| 16 | 13 | 52 | 9 | 9 | Clear, slightly | Soaked into the |
| | | | | | viscous | dish cloth |
| 18 | 22 | 50 | 10 | 0 | Clear solution | Gel on the |
| | | | | | | surface of the |
| | | | | | | dish cloth |

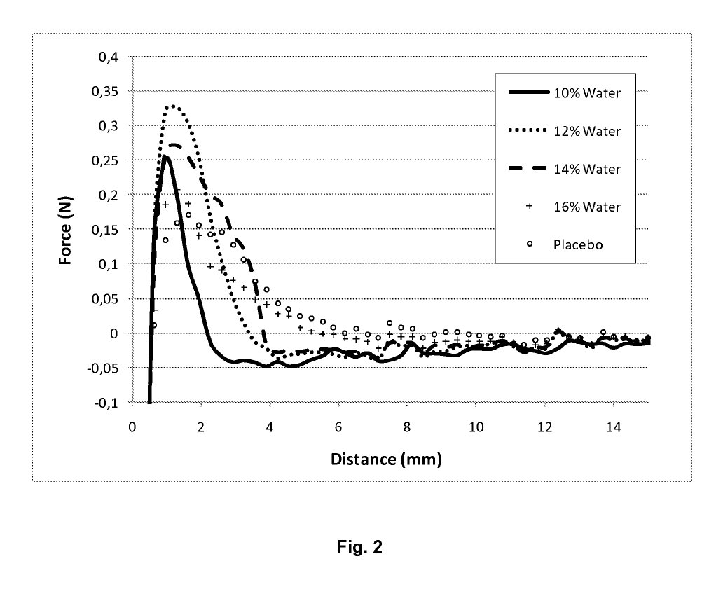

TABLE 13
Formulations used for mucoadhesion test. The results are shown in FIG. 2.

| | | | Oleic | | | |
|---|---|---|---|---|---|---|
| Ropivacain | Tween | GMO | acid | Water | NaOH | |
| (%) | (%) | (%) | (%) | (%) | (M) | Appearance |
| 3 | 10 | 52 | 25 | 10 | 2.5 | Viscous |
| | | | | | | solution |
| 3 | 10 | 50 | 25 | 12 | 2.1 | Viscous |
| | | | | | | solution |
| 3 | 10 | 49 | 24 | 14 | 1.9 | Viscous |
| | | | | | | solution |
| 3 | 10 | 48 | 23 | 16 | 1.6 | Gel (lamellar) |
| — | 10 | 47 | 24 | 16 | 2.5 | Gel (lamellar) |

The mucoadhesion measurements on 3% ropivacain formulations were performed on a Slip & Peel tester (SP2000 Imass, USA) by mounting a piece of porous cellulose substrate that was pre-soaked in 50 mM phosphate buffer (pH 5.0) between two clamping holder). 2 ml of the 3% ropivacain formulation (or 1 ml for the 3% ropivacain formulation with 16% water concentration and a placebo formulation) was applied on the whole soaked substrate and the formulation was allowed to swell on the substrate for 30 minutes before the measurement was started.

When the measurement started, the surfaces on the substrate were pressed together and then separated with a speed of 12.5 mm/s. During the separation of the surfaces, the adhesion force was recorded as a function of distance as shown in FIG. 2. It was not possible to correlate the maximum recorded adhesion force for each formulation with the water concentration in the formulations. Instead, a different method was used to evaluate the degree of mucoadhesion in the samples by analyzing the area below each area. This area represents the magnitude of the adhesion force, i.e. a larger area represents a formulation with large degree of mucoadhesion. The area under the force-distance curves in FIG. 2 was calculated in order to be able to visualize the degree of mucoadhesion in 3% ropivacain formulations with varying degree of water concentration. The area calculation results demonstrate that the formulations with higher water concentration has a larger area, which corresponds to a higher a degree of mucoadhesion between the ropivacain formulation and the porous cellulose substrate.

The results of Example 7 confirm the capacitive of the inventive compositions to swell at an aqueous administration site and establish bioadhesvive (mucoadhesive) characteristics. This is an important feature for the clinical performance of the composition in order to exert the anaesthetic effect over a controlled time period.

### Example 8

### In-Vitro Release of Ropivacaine From Pharmaceutical Compositions

Release of ropivacaine from pharmaceutical compositions according to Table 14 prepared as described above was measured overtime.

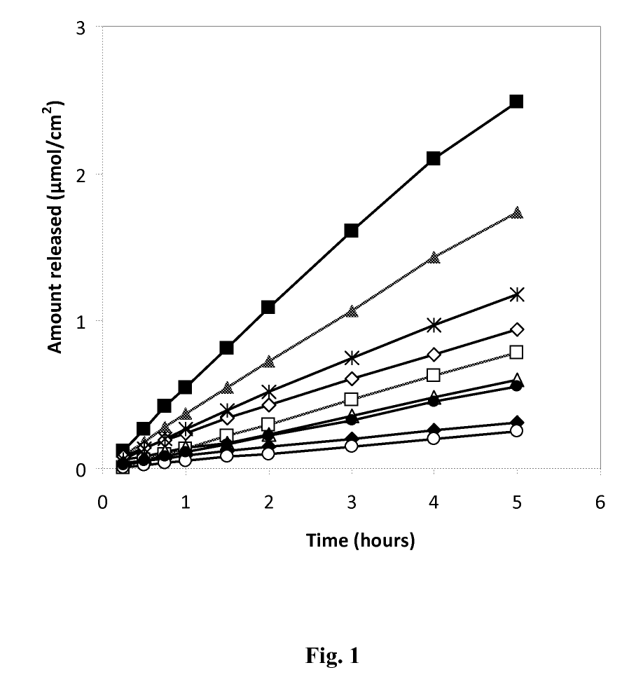

TABLE 14
Release of ropivacaine from pharmaceutical compositions

| Component | 1 | 2 | 3 | 4 | 5 | 6 | 7 | 8 | 9 |
|---|---|---|---|---|---|---|---|---|---|
| Ropivaccaine | 10% | 15% | 10% | 15% | 10% | 5% | 5% | 5% | 8% |
| GMO | 28% | 26% | 12% | 19% | 21% | 30% | 22% | 16% | 31% |
| Na-oleate | 42% | 39% | 18% | 46% | 49% | 45% | 33% | 24% | 46% |
| Glycerol formal | 10% | 10% | 10% | 10% | 10% | 10% | 10% | 5% | 0% |
| Water | 10% | 10% | 50% | 10% | 10% | 10% | 30% | 50% | 15% |
| Symbol FIG. 1 | -□- | -▪- | -Δ- | -▴- | -⋄- | -♦- | -◯- | -- | -*- |

Results are presented in FIG. 1. A steady release of ropivacaine could be observed from the different pharmaceutical preparations. The rate of release was found to be essentially related to the concentration of ropivacaine in the composition.

### Example 9

### Sterilization of the Pharmaceutical Compositions

In order to assess if the compositions according to invention was sufficiently stable to be heat sterilized without precipation or loss of essential characteristics autoclaving was performed in a CertoClav RO122259 (Austria) with valves for 125/140° C. and 115/121° C.

TABLE 15
All formulations contain 40/60 GMO/oleic acid and 10% Tween 80.
Autoclaving was performed in a CertoClav RO 122250 (Austria)
with valves for125/140° C. and 115/121° C.

| | Water | | Appearance | Appearance |
|---|---|---|---|---|
| Ropivacain | content | Autoclave | before | after |
| conc (%) | (%) | conditions | sterilisation | sterilisation |
| 3 | 10 | 110° C., 10 | Yellow viscous | Yellow viscous |
| | | min | solution | solution |
| 3 | 10 | 110° C., 15 | Yellow viscous | Yellow viscous |
| | | min | solution | solution |
| 3 | 10 | 121° C., 15 | Yellow viscous | Yellow viscous |
| | | min | solution | solution |
| 5 | 10 | 110° C., 10 | Yellow viscous | Yellow (slightly |
| | | min | solution | darker) viscous |
| | | | | solution |
| 5 | 10 | 110° C., 15 | Yellow viscous | Yellow (slightly |
| | | min | solution | darker) viscous |
| | | | | solution |
| 5 | 10 | 121° C., 15 | Yellow viscous | Yellow (slightly |
| | | min | solution | darker) viscous |
| | | | | solution |
| 10 | 10 | 110° C., 10 | Yellow viscous | Yellow (slightly |
| | | min | solution | darker) viscous |
| | | | | solution |
| 10 | 10 | 110° C., 15 | Yellow viscous | Yellow (slightly |
| | | min | solution | darker) viscous |
| | | | | solution |
| 10 | 10 | 121° C., 15 | Yellow viscous | Orange viscous |
| | | min | solution | solution |
| 10 | 15.5 | 121° C., 15 | Yellow, solid gel | Orange, solid |
| | | min | (lamellar) | gel (lamellar) |

The results of Table 15 confirm that the compositions were sufficiently stable.

### Example 10

### Sterilization with Different Autoclave Conditions

Spores of Geobacillus searothermophilus (ATCC 7953) were added in different amounts to the composition (308 mg/g glycerol monooleate, 432 mg/g oleic acid,100 mg/g Tween 80, 30 mg/g ropivacaine, 100 mg/g 2.57 M NaOH,

TABLE 16
Number of viable microorganisms

| | Amount of added spores (CFU/ml) | | | |
|---|---|---|---|---|
| Autoclave conditions | 10¹ | 10² | 10³ | 10⁴ |
| 105°/10 min | <5 | <5 | <5 | <5 |
| 110°/10 min | <5 | <5 | <5 | <5 |
| 115°/10 min | <5 | <5 | <5 | <5 |

The results of Table 16 indicate that the compositions according to the invention exhibit a surprisingly efficient capacity to reduce bacterial spores also at as low temperatures as 105° C.

Although particular embodiments have been disclosed herein in detail, this has been done by way of example for purposes of illustration only, and is not intended to be limiting with respect to the scope of the appended claims that follow. In particular, it is contemplated by the inventor that various substitutions, alterations, and modifications may be made to the invention without departing from the spirit and scope of the invention as defined by the claims.

### Example 11

### Further Investigation of the Compositions with XRD

Compositions according to the invention were studies with XRD to investigate their phase behaviour.

XRD powder patterns (PANalytical X'Pert PRO, The Netherlands) were obtained with a 3050/60 theta/theta goniometer and a PW3064 spinning stage. CuKa radiation (λ=1.5418 Å) was used in all experiments and the generator was operated at 45 kV and 35 mA. The powder was placed in the centre on the rotating sample holder and a diffractogram in the 2θ range 0.5-25° was obtained with step size 0.033°.

TABLE 17
Phase behavior ropivaciane formulations analyzed with XRD

| | | | | | | Konc | |
|---|---|---|---|---|---|---|---|
| Sample | | Oleic Acid | Tween 80 | Ropivacain | Water | NaOH | XRD |
| no | GMO (%) | (%) | (%) | (%) | (%) | (M) | results |
| 11-42 | GMO (28.0) | Oleic acid | Tween 80 | 3.1 | 16.8 | 1.50 | Mixture |
| | | (42.1) | (10.0) | | | | of |
| | | | | | | | phases, |
| | | | | | | | micellar |
| | | | | | | | solution + |
| | | | | | | | hexagonal |
| | | | | | | | or |
| | | | | | | | lamellar |
| | | | | | | | (weak) |
| 11-63 | GMO/GDO | Oleic acid | Tween 80 | 9.3 | 19.4 | 1.46-1.79 | Phase |
| | 60/40 | (36.9) | (10.4) | | (−21.6) | | separation, |
| | (24.8) | | | | | | micellar |
| | | | | | | | solution + |
| | | | | | | | hexagonal |
| | | | | | | | phase |
| 11-64 | GMS (29.5) | Oleic acid | Tween 80 | 3.0 | 13.1 | 2.13-2.15 | Lamellar |
| | | (44.0) | (10.9) | | (−15.1) | | phase |
| 11-67 | GML (26.3) | Oleic acid | Tween 80 | 9.9 | 14.1 | 1.57-1.73 | Micellar |
| | | (39.3) | (9.5) | | (15.0) | | solution + |
| | | | | | | | hexagonal |
| | | | | | | | phase |

The four investigated formulations all include hexagonal and/or lamellar phases which indicate that they have a capacity to swell at in an aqueous environment.

1. An aqueous stabilized pharmaceutical bioadhesive gelling composition comprising;
(a) an anaesthetically effective amount of one or more local anaesthetics;
(b) a monoglyceride or a diglyceride, or mixtures thereof of a long chain fatty acid in an amount of between 15 to 70% by weight; and
(c) a free long chain saturated or unsaturated fatty acid in an amount of between 5 to 60% by weight, wherein the composition has an anisotropic organic phase behaviour that admits swelling at administration site with excess water.

2. The pharmaceutical composition according to claim 1 further comprising;
(d) one or more solubilizer in an amount of between 0 to 30% by weight, preferably between 5 to 25% by weight and most preferably between 5 to 15% by weight.

3. The pharmaceutical composition according to claim 1 wherein the one or more local anaesthetics are present in an amount of between 0.1 to 20% by weight, preferably in an amount of between 0.5 to 12% by weight, most preferably in an amount of between 2 to 10% by weight.

4. The pharmaceutical composition according to claim 1 wherein the one or more local anaesthetic is a local anaesthetic of the amide type, ATC code N01BB.

5. The pharmaceutical composition according to claim 4 wherein the local anaesthetic of the amide type is selected from lidocaine, prilocaine, mepivacaine, ropivacaine, bupivacaine, and levobupivacaine.

6. The pharmaceutical composition according to claim 1 wherein the one or more local anaesthetic is a local anaesthetic of the ester type, ATC code N01BA.

7. The pharmaceutical composition according to claim 6 wherein the local anaesthetic of the ester type is selected from the group consisting of benzocaine, tetracaine, and chloroprocaine.

8. The pharmaceutical composition according to claim 1 wherein the one or more local anaesthetic is a long acting local anaesthetic.

9. The pharmaceutical composition according to claim 8 wherein the long acting local anaesthetic is selected from the group consisting of ropivacaine, bupivacaine, and levobupivacaine, preferably, the local anaesthetic is ropivaciane.

10. The pharmaceutical composition according to claim 1 wherein the one or more local anaesthetic is a short acting local anaesthetic.

11. The pharmaceutical composition according to claim 10 wherein the short acting local anaesthetic is selected from the group consisting of lidocaine, prilocaine, and mepivacaine.

12. The pharmaceutical composition according to claim 1 wherein the total amount of monoglycerides or diglyceride and free fatty acids together is more than 50% by weight in the composition, preferably between 50 to 75% by weight.

13. The pharmaceutical composition according to claim 1, wherein the content of water is less than 30% by weight, preferably, between 5 to 20% by weight.

14. The pharmaceutical composition according to claim 1, wherein the monoglycerides and/or diglyceride are present in an amount of 20 to 50% by weight.

15. The pharmaceutical composition according to claim 1 wherein monoglyceride is glycerol monooleate

16. The pharmaceutical composition according to claim 1 wherein the one or more fatty acids are present in an amount of between 15 to 70% by weight, preferably in an amount of between 25 to 50% by weight.

17. The pharmaceutical composition according to claim 1, wherein the fatty acid is selected among long-chain unsaturated fatty acids, preferably single unsaturated fatty acids, most preferably the fatty acids are selected among oleic acid and ricinoleic acid.

18. The pharmaceutical composition according to claim 1, wherein the fatty acid is selected among long-chain saturated fatty acids, most preferably the fatty acids are selected among palmitic acid and stearic acid.

19. The pharmaceutical composition according to claim 2 wherein the solubilizer is selected from the group consisting of non-ionic surfactants, preferably polysorbates or sorbitan fatty acid esters, glycerol formal, a polyoxyethylated castor oil (such as Cremophor EL).

20. The pharmaceutical composition according to claim 19, wherein the solubilizer is of the polysorbate type or a polyoxyethylated castor oil.

21. The pharmaceutical composition according to claim 1 wherein the final pH-value for the composition is higher or equal to the pKa of the local anaesthetic minus 1.0, preferably the final pH-value for the composition is higher or equal to the pKa of the local anaesthetic minus 0.5, even more preferably the final pH-value for the composition is higher or equal to the pKa of the local anaesthetic.

22. The pharmaceutical composition according to claim 1 comprising;
ropivacaine in an amount of between 3 to 10% by weight;
glycerol monooleate in an amount of between 40 to 70% by weight;
oleic acid or ricinoleic acid in an amount of between 15 to 30% by weight; and
a solubilizer in an amount of between 10 to 20% by weight.

23. A composition according to claim 22, comprising water in an amount between 10 and 20% by weight that is essentially semi-solid or solid at body temperature.

24. A method of preparing a gelling bioadhesive pharmaceutical composition
capable of exerting a long term anaesthetic effect in an aqueous environment comprising the consecutive steps of:
(a) providing a mixture of a monoglyceride of long-chain unsaturated fatty acid, a free long-chain fatty acid and a solubilizer for a local anesthetic;
(b) adding a local anaesthetic to the mixture of step (a);
(c) adding a water at a basic pH to the mixture of step (b); and
(d) obtaining a gelling composition with an isotropic organic phase behaviour that admits swelling at an administration site with excess water.

25. A method of manufacturing a stabilized local anaesthetic product with such a low level of viable microorganisms that the product is suitable for topical administration to an internal body site, comprising the steps of:
a) providing a composition of a local anaesthetic in a concentration of between 1 to 10% by weight and solubilized with at least 5% of a solubilizer, the composition further comprising at least 50% by weight of a monoglyceride or a diglyceride, or mixtures thereof of together with a long chain free fatty acid;
b) preparing a sealed container comprising the composition;
c) subjecting the container with the composition to heat sterilization (autoclavation) at less than 120° C. for about 10 minutes and;
d) obtaining a local anaesthetic product with maintained gelling characteristics and with so low level of viable microorganisms that the product is suitable for topical administration to an internal body site.

26. A method according to claim 24, wherein the monoglycerides and the fatty acid together is included to more than 50% by weight, preferably between 50 to 75% by weight, in the resulting composition; and wherein the water content is between 5 to 20% by weight in the resulting composition.

27. A method according to claim 24, wherein the monoglycerides is glycerol monooleate and the fatty acid is oleic acid.

28. A method according to claim 24, wherein the solubilizer is of a polysorbate, a sorbitan fatty acid ester or a polyoxyethylated castor oil and the local anaesthetic is ropivacaine.
